(19) United States
(12) Patent Application Publication
(10) Pub. No.: US 2010/0029665 A1
(43) Pub. Date: Feb. 4, 2010

(54) Methods and Compositions for Treating Migraine Pain

(76) Inventors: Laurence R. Meyerson, Las Vegas, NV (US); Gregory T. Went, Mill Valley, CA (US); Timothy J. Fultz, Pleasant Hill, CA (US); Timothy S. Burkoth, San Francisco, CA (US)

Correspondence Address: Adamas Pharmaceuticals, Inc., 1900 Powell Street, Suite 1050, Emeryville, CA, 94608, US

(21) Appl. No.: 12/420,790

(22) Filed: Apr. 8, 2009

Related U.S. Application Data
(63) Continuation of application No. 11/247,775, filed on Oct. 11, 2005, now abandoned.
(60) Provisional application No. 60/617,238, filed on Oct. 8, 2004.

(51) Int. Cl. A61K 31/13; A61K 31/138; A61K 31/437; A61K 31/404; A61K 31/403; A61K 31/4985; A61P 25/06

(52) U.S. Cl. 514/250; 514/662; 514/654; 514/288; 514/415; 514/411; 514/414

(57) ABSTRACT

The present invention provides novel methods and compositions for the treatment and prevention of headaches, vascular headaches, migraine headaches, cluster headaches, and migraine. One of the headaches, vascular headaches, migraine headaches, cluster headaches, and migraine treated by the methods and compositions of the invention is migraine.

### RELATED APPLICATION

This application claims priority to U.S. Ser. No. 60/617,238, filed Oct. 8, 2004. The content of this application is incorporated herein by reference in its entirety.

### FIELD OF THE INVENTION

This invention relates to methods and compositions for treating and reducing a migraine or headache.

### BACKGROUND OF THE INVENTION

There are two major types of migraines. The common migraine affects 80-85% of migraine sufferers and classical migraine with aura affects 15% of migraine sufferers. The common migraine is typically associated with various psychological (e.g., irritability, depression, fatigue, drowsiness, and restlessness), neurological (e.g., photophobia, and phonophobia), and gastrointestinal symptoms. The headache starts with mild pain, which increases in intensity over a short period of time. In some cases, early management of the headache can reduce the duration and severity of the pain. Headaches in classical migraines are typically characterized by a neurological deficit known as an aura. Exemplary deficits include visual scotoma or visual designs, hemiplegia, migrating paraesthesia, dysarthria, dysphasia, and deja-vus. The headache is usually accompanied by light or sound sensitivity, photophobia or phonophobia, irritability and impaired concentration. Treatment of the classical migraine at the time of the aura may alleviate the severity and duration of the headache.

Currently available drugs to alleviate the pain associated with migraines have modest or limited efficacy and are associated with various debilitating side effects. Thus, better therapies are needed for the management of migraines.

### SUMMARY OF THE INVENTION

In general, the present invention provides methods and compositions for treating and preventing a migraine or other headache (e.g., vascular headache and cluster headache) by administering to a subject in need thereof a combination that includes an NMDA receptor antagonist and a second agent such as a beta adrenergic antagonist (e.g., propranolol, atenolol, and nadolol), serotonin antagonist (e.g., methysergide), steroid (e.g., prednisone), serotonin receptor agonist (e.g., triptan such as frovatriptan, sumatriptan, zolmitriptan, rizatriptan, naratriptan, and eletriptan; ergotamine; and dihydroergotamine (DHE)), verapamil, or botulinum toxin. The administration of the combinations described herein results in the alleviation and prevention of the migraine or headache, which may be associated with or arise from any CNS-related conditions and may be associated with loss of memory, loss of balance, hallucinations, delusions, agitation, withdrawal, depression, communication problems, cognitive loss, personality change, confusion and insomnia. The combinations of the present invention may also be used in the prevention, reduction, or treatment of pain associated with migraines or headaches and may also be helpful for the treatment and prevention of cerebrovascular diseases, motor neuron diseases, dementias, neurodegenerative diseases, strokes, movement disorders, ataxic syndromes, disorders of the sympathetic nervous system, cranial nerve disorders, myelopathies, traumatic brain and spinal cord injuries, radiation brain injuries, multiple sclerosis, post-meningitis syndrome, prion diseases, myelitic disorders, radiculitis, neuropathies, pain syndromes, axonic brain damage, encephalopathies, chronic fatigue syndrome, psychiatric disorders, glucose dysregulation, and drug dependence.

The NMDA receptor antagonist, the second agent, or both agents may be administered in an amount similar to that typically administered to subjects. Optionally, the amount of the NMDA receptor antagonist, the second agent, or both agents may be administered in an amount greater than or less than the amount that is typically administered to subjects. If desired, the amount of the NMDA receptor antagonist in the pharmaceutical composition is less than the amount of NMDA receptor antagonist required in a unit dose to obtain the same therapeutic effect for treating or reducing pain when the NMDA receptor antagonist is administered in the absence of the second agent. Alternatively, the amount of the second agent in the pharmaceutical composition is less than the amount of the second agent required in a unit dose to obtain the same therapeutic effect for treating or reducing the migraine or headache when the second agent is administered in the absence of the NMDA receptor antagonist. Optionally, the NMDA receptor antagonist, the NMDA receptor antagonist, or both are present at a higher dose than that typically administered to a subject for a specific condition. For example, the amount of memantine required to positively affect the patient response (inclusive of adverse effects) may be 2.5-80 mg per day rather than the typical 10-20 mg per day administered for presently approved indications i.e. without the improved formulation described herein. A higher dose amount of the NMDA receptor antagonist in the present invention may be employed whereas a lower dose of the NMDA receptor antagonist may be sufficient when combined with the second agent to achieve a therapeutic effect in the patient. Optionally, lower or reduced amounts of both the NMDA receptor antagonist and the second agent are used in a unit dose relative to the amount of each agent when administered as a monotherapy.

The invention also provides a pharmaceutical composition that includes an NMDA receptor antagonist and a second agent such as a beta adrenergic antagonist (e.g., propranolol, atenolol, and nadolol), serotonin antagonist (e.g., methysergide), steroid (e.g., prednisone), serotonin receptor agonists (e.g., triptan such as frovatriptan, sumatriptan, zolmitriptan, rizatriptan, naratriptan, and eletriptan; ergotamine; and dihydroergotamine (DHE)), verapamil, or botulinum toxin. Optionally, a pharmaceutically acceptable carrier is included.

The NMDA receptor antagonist, the second agent, or both agents may be provided in a controlled or extended release form with or without an immediate release component in order to maximize the therapeutic benefit of each, while reducing unwanted side effects associated with each. When these drugs are provided in an oral form without the benefit of controlled or extended release components, they are released and transported into the body fluids over a period of minutes to several hours. Thus, the composition of the invention may contain an NMDA receptor antagonist and a sustained release component, such as a coated sustained release matrix, a sustained release matrix, or a sustained release bead matrix. In one example, memantine (e.g., 5-80 mg) is formulated without an immediate release component using a polymer matrix (e.g., Eudragit), Hydroxypropyl methyl cellulose (HPMC) and a polymer coating (e.g., Eudragit). Such formulations are comprised into solid tablets or granules. Optionally, a coating such as Opydry® or Surelease® is used.

Optionally, the composition described herein is formulated such the N-methyl-D-aspartate (NMDA) receptor antagonist or the second agent has an in vitro dissolution profile less than 40% in one hour, less than 70% in four hours, between 1% and 80% in 6 hours, 30% and 90% in 10 hours, and 60% and 100% in 12 hours and greater than 84% in 16 hours using, for example, a USP type 2 (paddle) dissolution system at 50 rpm, at a temperature of 37±0.5° C. with 0.1N HCl as a dissolution medium. Alternatively, the N-methyl-D-aspartate (NMDA) receptor antagonist or the second agent has an in vitro dissolution profile in a solution with a neutral pH (e.g., water) that is substantially the same as its dissolution profile in an acidic dissolution medium (see FIG. 1A).

As another example, the composition described herein is formulated such the N-methyl-D-aspartate (NMDA) receptor antagonist or the second agent has an in vitro dissolution profile ranging between 0.1%-20% in one hour, 5%-30% in two hours, 40%-80% in six hours, 50%-90% in 10 hours, and 90%-95% in 12 hours using, for example, a USP type 2 (paddle) dissolution system at 50 rpm, at a temperature of 37±0.5° C. with 0.1N HCl as a dissolution medium. Alternatively, the N-methyl-D-aspartate (NMDA) receptor antagonist or the second agent has an in vitro dissolution profile in a solution with a neutral pH (e.g., water) that is substantially the same as its dissolution profile in an acidic dissolution medium. Thus, the NMDA receptor antagonist or the second agent may be released at the following rate: between 0.1-20% in one hour, 5-30% in two hours, 40-80% in six hours, 70-90% in 10 hours, and 90%-95% in 12 hours as obtained using a USP type II (paddle) dissolution system at 50 rpm, at a temperature of 37±0.5° C.

As used herein, “C” refers to the concentration of an active pharmaceutical ingredient in a biological sample, such as a patient sample (e.g. blood, serum, and cerebrospinal fluid). The concentration of the drug in the biological may be determined by any standard assay method known in the art. The term “Cmax” refers to the maximum concentration reached by a given dose of drug in a biological sample. The term “Cmean” refers to the average concentration of the drug in the sample over time. Cmax and Cmean may be further defined to refer to specific time periods relative to administration of the drug. The time required to reach the maximal concentration (“Cmax”) in a particular patient sample type is referred to as the “Tmax.” The agents of the combination are administered in formulations that reduce the variability of the ratio of the concentrations of the active agents over a period of time, thereby maximizing the therapeutic benefit while minimizing the side effects.

In a preferred embodiment, the dosage form is provided in a non-dose escalating, twice per day or once per day form. In such cases, the concentration ramp (or Tmax effect) may be reduced so that the change in concentration as a function of time (dC/dT) is altered to reduce or eliminate the need to dose escalate the drug. A reduction in dC/dT may be accomplished, for example, by increasing the Tmax in a relatively proportional manner. Accordingly, a two-fold increase in the Tmax value may reduce dC/dT by approximately a factor of 2. Thus, the NMDA receptor antagonist may be provided so that it is released at a rate that is significantly reduced over an immediate release (so called IR) dosage form, with an associated delay in the Tmax. The pharmaceutical composition may be formulated to provide a shift in Tmax by 24 hours, 16 hours, 8 hours, 4 hours, 2 hours, or at least 1 hour. The associated reduction in dC/dT may be by a factor of approximately 0.05, 0.10, 0.25, 0.5, or at least 0.8. In certain embodiments, this is accomplished by releasing less than 30%, 50%, 75%, 90%, or 95% of the NMDA receptor antagonist into the circulatory or neural system within one hour of such administration.

Optionally, the sustained release formulations exhibit plasma concentration curves having initial (e.g., from 2 hours after administration to 4 hours after administration) slopes less than 75%, 50%, 40%, 30%, 20% or 10% of those for an IR formulation of the same dosage of the same NMDA receptor antagonist. The precise slope for a given individual will vary according to the NMDA receptor antagonist being used, the quantity delivered, or other factors, including, for some active pharmaceutical agents, whether the patient has eaten or not. For other doses, e.g., those mentioned above, the slopes vary directly in relationship to dose.

Using the sustained release formulations described herein, the NMDA receptor antagonist or the second agent reaches a therapeutically effective steady state plasma concentration in a subject within the course of the first five, seven, nine, ten, twelve, fifteen, or twenty days of administration. For example, the formulations described herein, when administered at a substantially constant daily dose (e.g., at a dose ranging between 15 mg and 35 mg and preferably between 20 and 25 mg) may reach a steady state plasma concentration in approximately 70%, 60%, 50%, 40%, 30%, or less of the time required to reach such plasma concentration when using a dose escalating regimen.

The ratio of the concentrations of two agents in a combination is referred to as the “Cratio,” which may fluctuate as the combination of drugs is released, transported into the circulatory system or CNS, metabolized, and eliminated. An objective of the present invention is to stabilize the Cratio for the combinations described herein. In some embodiments, the variation in the Cratio (termed “Cratio,var”) is as low as possible.

The present invention therefore features formulations of combinations directed to dose optimization or release modification to reduce adverse effects associated with separate administration of each agent. The combination of the NMDA receptor antagonist and the second agent may result in an additive or synergistic response, as described below.

If desired, the NMDA receptor antagonist or the second agent of the combination is released into a subject sample at a slower rate than observed for an immediate release (IR) formulation of the same quantity of the antagonist. The release rate is measured as the dC/dT over a defined period within the period of 0 to Tmax for the IR formulation and the dC/dT rate is less than about 80% of the rate for the IR formulation. In some embodiments, the dC/dT rate is less than about 60%, 50%, 40%, 30%, 20% or 10% of the rate for the IR formulation. Similarly, the second agent may also be released into a patient sample at a slower rate than observed for an IR formulation of the same quantity wherein the release rate is measured as the dC/dT over a defined period within the period of 0 to Tmax for the IR formulation and the dC/dT rate is less than about 80%, 60%, 50%, 40%, 30%, 20%, or 10%, of the rate for the IR formulation of the same NMDA receptor antagonist over the first 1, 2, 4, 6, 8, 10, or 12 hours.

In all foregoing aspects of the invention, at least 50%, 80, 90%, 95%, or essentially all of the NMDA receptor antagonist in the pharmaceutical composition may be provided in a controlled release dosage form. In some embodiments, at least 99% of the NMDA receptor antagonist remains in the extended dosage form one hour following introduction of the pharmaceutical composition into a subject. The NMDA receptor antagonist may have a Cₘₐₓ/Cₘₑₐₙ of approximately 2, 1.6, 1.5, 1.4, 1.3, 1.2 or less, approximately 2 hours to at least 8, 12, 16, 24 hours after the NMDA receptor antagonist is introduced into a subject. The second agent may also be provided in a controlled release dosage form. Thus, at least 50%, 60%, 70%, 80%, 90%, 95%, or essentially all of the second agent may be provided as a controlled release formulation. If provided as such, the second agent may have a Cₘₐₓ/Cₘₑₐₙ of approximately 2, 1.6, 1.5, 1.4, 1.3, 1.2 or less, approximately 2 hours to at least 6, 8, 12, 16, or 24 hours after the second agent is introduced into a subject.

The active pharmaceutical agents may be administered to the patient in a manner that reduces the variability of the ratio of the concentrations of the active agents over a period of time, thereby maximizing the therapeutic benefit while minimizing the side effects. The present invention differs from prior studies by providing novel combinations as well as formulations of combinations directed to dose optimization or release modification to reduce adverse effects associated with each agent.

Optionally, the Cratio,var of the NMDA receptor antagonist and the second agent is less than 100%, e.g., less than 70%, 50%, 30%, 20%, or 10% after the agents have reached steady-state conditions. Optionally, the Cratio,var of the NMDA receptor antagonist and the second agent is less than 100%, e.g. less than 70%, 50%, 30%, 20%, or 10% during the first 24 hours post-administration of the agents. In some embodiments, the Cratio,var is less than about 90% (e.g., less than about 75% or 50%) of that for IR administration of the same active pharmaceutical ingredients over the first 4, 6, 8, or 12 hours after administration.

In all foregoing aspects of the invention, the NMDA receptor antagonist may be an aminoadamantine derivative including memantine (1-amino-3,5-dimethyladamantane), rimantadine (1-(1-aminoethyl)adamantane), or amantadine (1-amino-adamantane). The second agent may be a beta adrenergic antagonist (e.g., propranolol, atenolol, and nadolol), serotonin antagonist (e.g., methysergide), steroid (e.g., prednisone), serotonin receptor agonists (e.g., triptan such as frovatriptan, sumatriptan, zolmitriptan, rizatriptan, naratriptan, and eletriptan; ergotamine; and dihydroergotamine (DHE)), verapamil, or botulinum toxin.

In some embodiments, the NMDA receptor antagonist, the second agent, or both agents are formulated for oral, intravenous, topical, intranasal, subtopical transepithelial, subdermal, or inhalation delivery. Thus, the agents described herein may be formulated as a suspension, capsule, tablet, suppository, lotion, patch, or device (e.g., a subdermally implantable delivery device or an inhalation pump). If desired, the NMDA antagonist and the second agent may be admixed in a single composition. Alternatively, the two agents are delivered in separate formulations sequentially, or within one hour, two hours, three hours, six hours, 12 hours, or 24 hours of each other. If administered separately, the two agents may be administered by the same or different routes of administration three times a day, twice a day, once a day, or even once every two days. Optionally, the two agents are be provided together in the form of a kit. Preferably, the NMDA receptor antagonist and the second agent are provided in a unit dosage form.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting. All parts and percentages are by weight unless otherwise specified.

### BRIEF DESCRIPTION OF THE FIGURES

FIG. 1A is a graph showing the dissolution profiles of an immediate release formulation of memantine (Namenda) and sustained release formulations of memantine (NPI-6601, NPI-6701, and NPI-6801). The sustained release formulations contain 22.5 mg of memantine. These dissolution profiles were obtained from a USP II Paddle system using water as the medium.

FIG. 11B is a graph showing predicted plasma blood levels for 24 hours of dosing with an immediate release formulation of memantine (Namenda) and sustained release formulations of memantine (NPI-6601, NPI-6701, and NPI-6801), obtained using the Gastro-Plus software package v.4.0.2. The sustained release formulations contain 22.5 mg of memantine.

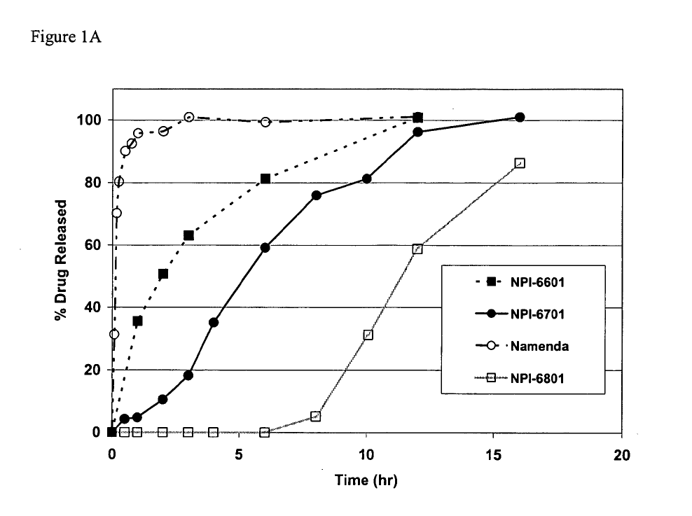

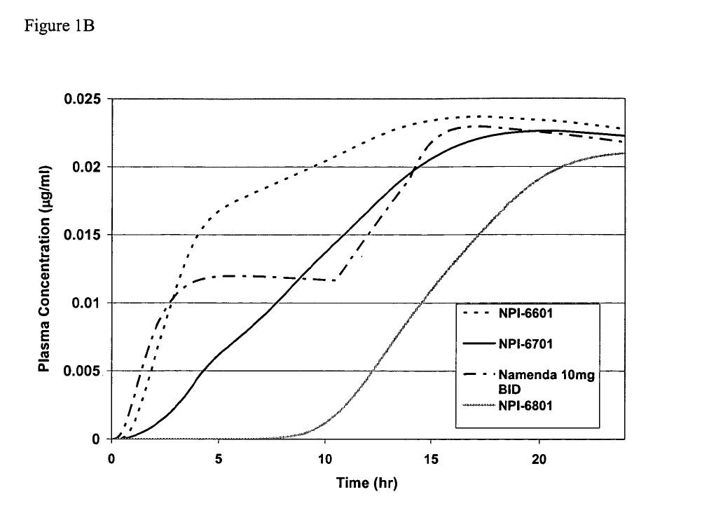

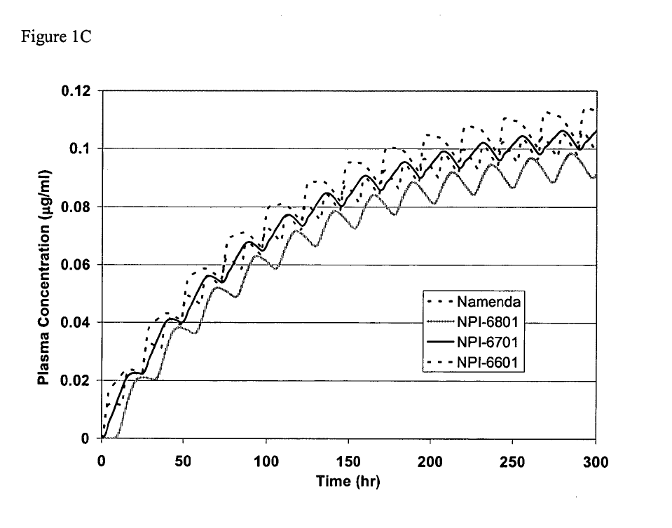

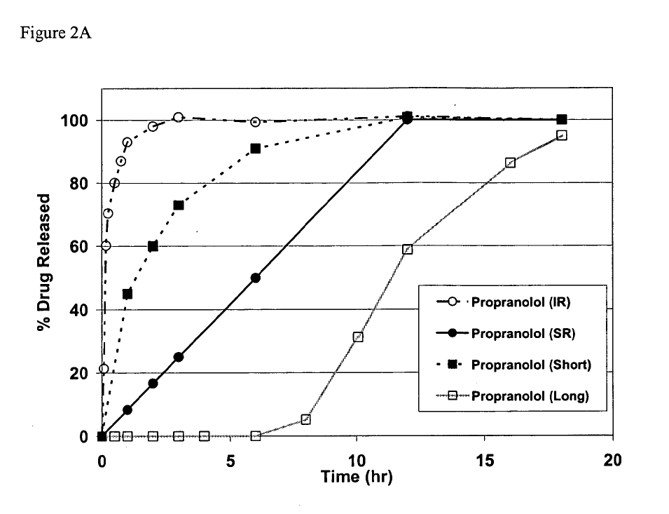

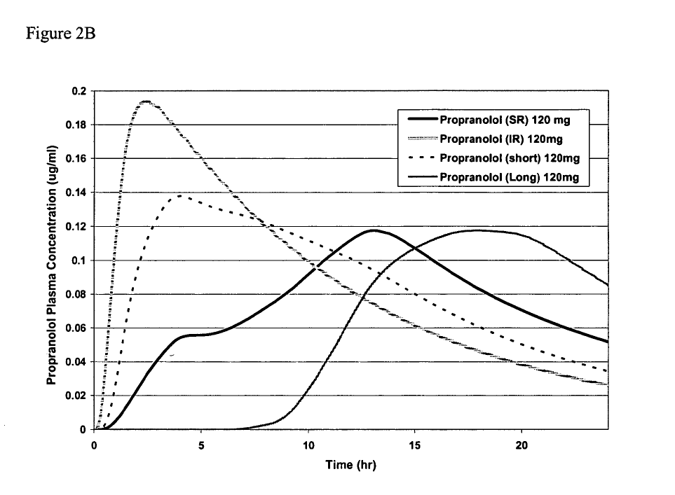

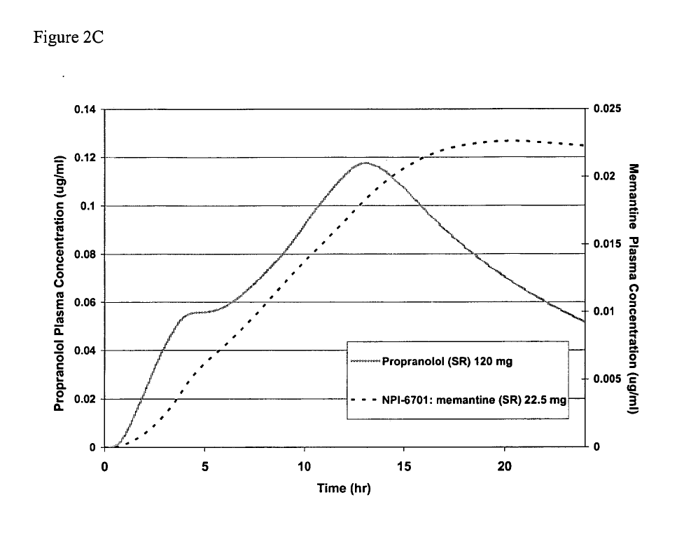

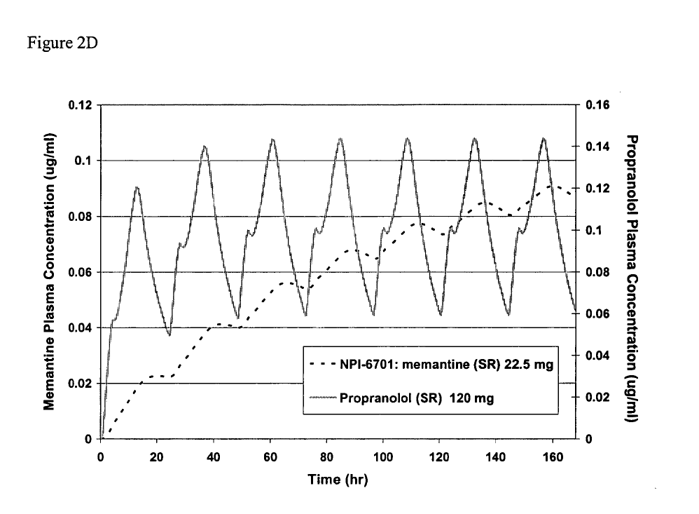

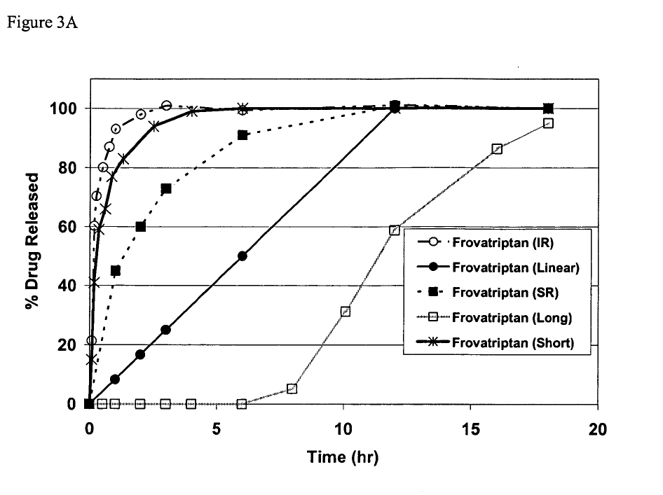

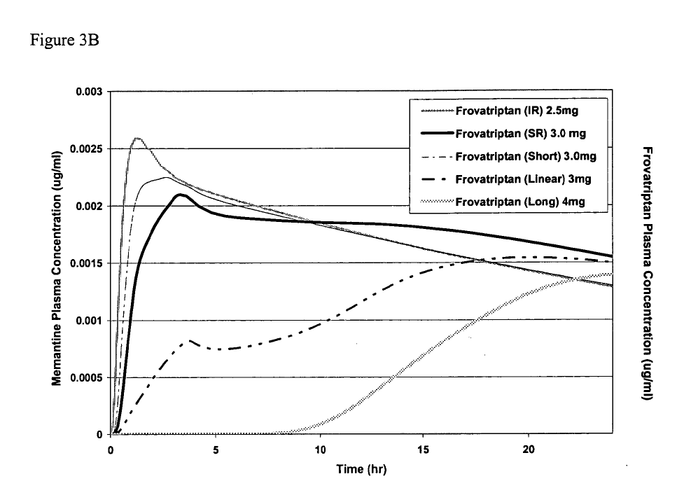

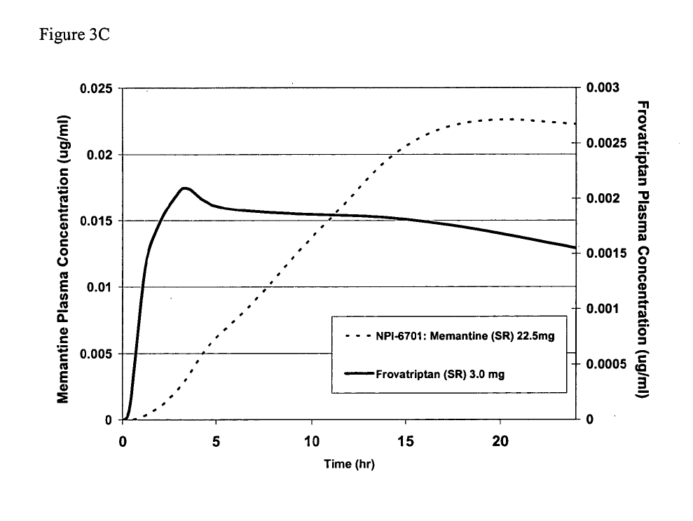

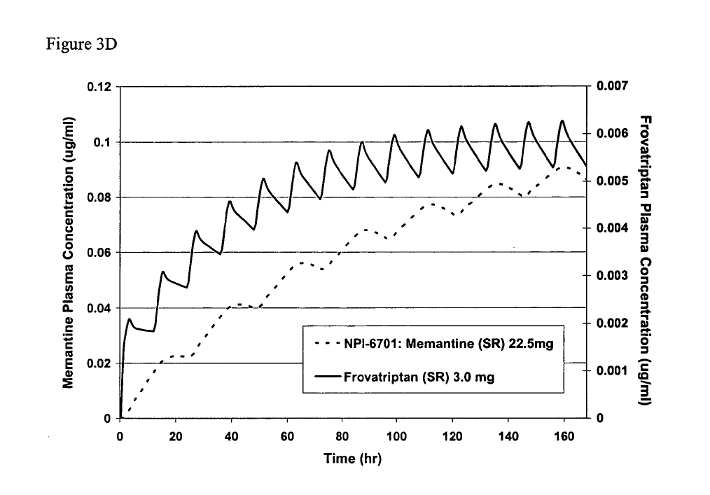

FIG. 1C is a graph predicting plasma blood levels at steady state for an immediate release formulation of memantine (Namenda) and sustained release formulations of memantine (NPI-6601, NPI-6701, and NPI-6801), obtained using the Gastro-Plus software package v.4.0.2. The sustained release formulations contain 22.5 mg of memantine.

FIG. 2A is a graph showing the dissolution profiles for propranolol sustained release (SR) Systems (120 mg) component of combination, obtained using the Gastro-Plus software package v.4.0.2

FIG. 2B is a graph showing the predicted plasma blood levels for 24 hours of dosing with Propranolol SR Systems component of combination, obtained using the Gastro-Plus software package v.4.0.2.

FIG. 2C is a graph showing the predicted plasma blood levels over 24 hours of dosing with Memantine and Propranolol SR combination, obtained using the Gastro-Plus software package v.4.0.2.

FIG. 2D is a graph showing the predicted plasma blood levels over multiple dosing using present NPI Memantine and Propranolol SR combinations, obtained using the Gastro-Plus software package v.4.0.2.

FIG. 3A is a graph showing the dissolution profiles for prophetic Frovatriptan SR Systems (3 mg) component of combination, obtained using the Gastro-Plus software package v.4.0.2.

FIG. 3B is a graph showing predicted plasma blood levels for 24 hours of dosing with Frovatriptan SR Systems component of combination, obtained using the Gastro-Plus software package v.4.0.2.

FIG. 3C is a graph showing predicted plasma blood levels over 24 hours of dosing with Memantine and Frovatriptan SR combination, obtained using the Gastro-Plus software package v.4.0.2.

FIG. 3D showing predicted plasma blood levels at steady state using NPI Frovatriptan SR Systems component and Memantine SR components of combinations, obtained using the Gastro-Plus software package v.4.0.2.

### DETAILED DESCRIPTION OF THE INVENTION

The present invention provides methods and compositions for treating or preventing a migraine or headache (e.g., vascular headache and cluster headache). The migraine or headache may be caused by glucose dysregulation, CNS-related conditions, including psychiatric disorders (e.g., panic syndrome, general anxiety disorder, phobic syndromes of all types, mania, manic depressive illness, hypomania, unipolar depression, depression, stress disorders, PTSD, somatoform disorders, personality disorders, psychosis, and schizophrenia), and drug dependence (e.g., alcohol, psychostimulants (e.g., crack, cocaine, speed, and meth), opioids, and nicotine), epilepsy, acute pain, chronic pain, neuropathies, cereborischemia, dementias, movement disorders, and multiple sclerosis. The combination includes a first agent that is an NMDA receptor antagonist and a second agent that is a beta adrenergic antagonist (e.g., propranolol, atenolol, and nadolol), serotonin antagonist (e.g., methysergide), steroid (e.g., prednisone), serotonin receptor agonist (e.g., triptan such as frovatriptan, sumatriptan, zolmitriptan, rizatriptan, naratriptan, and eletriptan; ergotamine; and dihydroergotamine (DHE)), verapamil, or botulinum toxin. The combination is administered such that the migraine (or pain associated therewith) or headache is reduced or prevented. Desirably, either of these two agents, or even both agents, is formulated for extended release, thereby providing a concentration and optimal concentration ratio over a desired time period that is high enough to be therapeutically effective but low enough to reduce or avoid adverse events associated with excessive levels of either agent in the subject.

### Making NMDA Receptor Antagonist Controlled Release Formulations

A pharmaceutical composition according to the invention is prepared by combining a desired NMDA receptor antagonist or antagonists with one or more additional ingredients that, when administered to a subject, causes the NMDA receptor antagonist to be released at a targeted concentration range for a specified period of time. A release profile, i.e., the extent of release of the NMDA receptor antagonist over a desired time, can be conveniently determined for a given time by calculating the Cₘₐₓ/Cₘₑₐₙ for a desired time range. For example, the NMDA receptor antagonist can be provided so that it is released at Cₘₐₓ/Cₘₑₐₙ of approximately 2 or less for approximately 2 hours to at least 6 hours after the NMDA receptor antagonist is introduced into a subject. One of ordinary skill in the art can prepare combinations with a desired release profile using the NMDA receptor antagonists and formulation methods described below. Optionally, the second agent may also be prepared as a controlled release formulation.

Using the formulations described herein, therapeutic levels may be achieved while minimizing debilitating side-effects that are usually associated with immediate release formulations. Furthermore, as a result of the reduction in the time to obtain peak plasma level and the potentially extended period of time at the therapeutically effective plasma level, the dosage frequency may be reduced to, for example, once or twice daily dosage, thereby improving patient compliance and adherence. For example, side effects including psychosis and cognitive deficits associated with the administration of NMDA receptor antagonists may be lessened in severity and frequency through the use of controlled-release methods that shift the Tmax to longer times, thereby reducing the dC/dT of the drug. Reducing the dC/dT of the drug not only increases Tmax, but also reduces the drug concentration at Tmax and reduces the Cmax/Cmean ratio providing a more constant amount of drug to the subject being treated over a given period of time and reducing adverse events associated with dosing.

### NMDA Receptor Antagonists

Any NMDA receptor antagonist can be used in the methods and compositions of the invention, particularly those that are non-toxic when used in the combination of the invention. The term “nontoxic” is used in a relative sense and is intended to designate any substance that has been approved by the United States Food and Drug Administration (“FDA”) for administration to humans or, in keeping with established regulatory criteria and practice, is susceptible to approval by the FDA or similar regulatory agency for any country for administration to humans or animals.

The NMDA receptor antagonist may be an amino-adamantane compound including, for example, memantine (1-amino-3,5-dimethyladamantane), rimantadine (1-(1-aminoethyl)adamantane), amantadine (1-amino-adamantane), as well as pharmaceutically acceptable salts thereof. Memantine is described, for example, in U.S. Pat. Nos. 3,391,142, 5,891,885, 5,919,826, and 6,187,338. Amantadine is described, for example, in U.S. Pat. Nos. 3,152,180, 5,891,885, 5,919,826, and 6,187,338. Additional aminoadamantane compounds are described, for example, in U.S. Pat. Nos. 4,346,112, 5,061,703, 5,334,618, 5,382,601, 6,444,702, 6,620,845, and 6,662,845. All of these patents are hereby incorporated by reference.

Further NMDA receptor antagonists that may be employed include, for example, ketamine, eliprodil, ifenprodil, dizocilpine, remacemide, iamotrigine, riluzole, aptiganel, phencyclidine, flupirtine, celfotel, felbamate, neramexane, spermine, spermidine, levemopamil, dextromethorphan ((+)-3-hydroxy-N-methylmorphinan) and its metabolite, dextrorphan ((+)-3-hydroxy-N-methylmorphinan), a pharmaceutically acceptable salt or ester thereof, or a metabolic precursor of any of the foregoing.

The NMDA receptor antagonist may be provided so that it is released at a dC/dT that is significantly reduced over an instant release (so called IR) dosage form, with an associated delay in the Tmax. The pharmaceutical composition may be formulated to provide a shift in Tmax by 24 hours, 16 hours, 8 hours, 4 hours, 2 hours, or at least 1 hour. The associated reduction in dC/dT may be by a factor of approximately 0.05, 0.10, 0.25, 0.5 or at least 0.8. In addition, the NMDA receptor antagonist may be provided such that it is released at rate resulting in a Cₘₐₓ/Cₘₑₐₙ of approximately 2 or less for approximately 2 hours to at least 8 hours after the NMDA receptor antagonist is introduced into a subject.

In addition, the NMDA receptor antagonist may be provided such that it is released at rate resulting in a Cₘₐₓ/Cₘₑₐₙ of approximately 2 or less for approximately 2 hours to at least 8 hours after the NMDA receptor antagonist is introduced into a subject. Optionally, the sustained release formulations exhibit plasma concentration curves having initial (e.g., from 2 hours after administration to 4 hours after administration) slopes less than 75%, 50%, 40%, 30%, 20% or 10% of those for an IR formulation of the same dosage of the same NMDA receptor antagonist. The precise slope for a given individual will vary according to the NMDA receptor antagonist being used or other factors, including whether the patient has eaten or not. For other doses, e.g., those mentioned above, the slopes vary directly in relationship to dose. The determination of initial slopes of plasma concentration is described, for example, by U.S. Pat. No. 6,913,768, hereby incorporated by reference.

Optionally, the composition described herein is formulated such the N-methyl-D-aspartate (NMDA) receptor antagonist has an in vitro dissolution profile ranging between 0.1%-20% in one hour, 5%-30% in two hours, 40%-80% in six hours, 50%-90% in 10 hours, and 90%-95% in 12 hours using a USP type 2 (paddle) dissolution system at 50 rpm, at a temperature of 37±0.5° C. with 0.1N HCl as a dissolution medium, for example. Desirably, the composition is formulated such that the NMDA receptor antagonist has an in vitro dissolution profile in a neutral dissolution medium that is substantially the same as its vitro dissolution profile in an acidic dissolution medium (e.g., HCl at pH 1.2). For example, the NMDA receptor antagonist may be released as follows: between 0.1-20% in one hour, 5-30% in two hours, 40-80% in six hours, 70-90% in 10 hours, and 90%-95% in 12 hours in a neutral and an acidic solution (e.g., in hydrochloride solution at a pH of 1.2) as measured using a USP type II (paddle) dissolution system at 50 rpm, at a temperature of 37±0.5° C.

Desirably, the compositions described herein have an in vitro profile that is substantially identical to the dissolution profile shown for the controlled release formulations shown in FIG. 1A, 2A, or 3A and, upon administration to a subject at a substantially constant daily dose, achieves a serum concentration profile that is substantially identical to that shown in FIGS. 1B and 2B.

The pharmaceutical composition may be formulated to provide memantine in an amount ranging between 1 and 80 mg/day, 5 and 40 mg/day, or 10 and 20 mg/day; amantadine in an amount ranging between 25 and 500 mg/day, 25 and 300 mg/day, or 100 and 300 mg/day; or dextromethorphan in an amount ranging between 1 and 5000 mg/day, 1 and 1000 mg/day, 100 and 800 mg/day, or 200 and 500 mg/day. Pediatric doses will typically be lower than those determined for adults. Representative dosing can be found in the PDR by anyone skilled in the art.

Table 1 shows exemplary the pharmacokinetic properties (e.g., Tmax and T1/2) of memantine, amantadine, and rimantadine

TABLE 1
Pharmacokinetics and Tox in humans for
selected NMDAr antagonists

| | Human | | | |
|---|---|---|---|---|
| | PK (t½) | Tmax in | Normal | Dose Dependent |
| Compound | in hrs | hrs | Dose | Tox |
| Memantine | 60 | 3 | 10-20 mg/day, | Dose escalation |
| | | | starting at 5 mg | required, |
| | | | | hallucination |
| Amantadine | 15 | 3 | 100-300 mg/day | Hallucination |
| Rimantadine | 25 | 6 | 100-200 mg/day | Insomnia |

### Second Agent

The second agent of the combination described herein may be a beta adrenergic antagonist (e.g., propranolol, atenolol, and nadolol), serotonin antagonist (e.g., methysergide), steroid (e.g., prednisone), serotonin receptor agonist (e.g., triptan such as frovatriptan, sumatriptan, zolmitriptan, rizatriptan, naratriptan, and eletriptan; ergotamine; and dihydroergotamine (DHE)), verapamil, or botulinum toxin. Normal therapeutic doses for most of these agents may be found in the Physician desk reference (PDR). Exemplary daily doses are provided below.

| | | |
|---|---|---|
| Propranolol | 160-240 | mg |
| Sumatriptan | 25-100 | mg |
| Frovatriptan | 1-7.5 | mg |
| Rizatriptan | 5-10 | mg |
| Naratriptan | 1-2.5 | mg |
| Eletriptan | 20-40 | mg |

In addition to the specific combinations disclosed herein, combinations made of a first NMDAr antagonist and the second agent may be identified by testing the ability of a test combination of a selected NMDAr antagonist and one or more second agents to lessen pain. Preferred combinations are those in which a lower therapeutically effective amount of the NMDA receptor antagonist and/or the second agent is present relative to the same amount of the NMDA receptor antagonist and/or the second agent required to obtain the same effect when each agent is tested separately.

The amounts and ratios of the NMDA receptor antagonist and the second agent are conveniently varied to maximize the therapeutic benefit and minimize the toxic or safety concerns. The NMDA receptor antagonist may range between 20% and 200% of its normal effective dose and the second agent may range between 20% to 200% of its normal effective dose. The precise ratio may vary according to the condition being treated. In one example, the amount of memantine ranges between 2.5 and 40 mg per day and the amount of eletriptan ranges between 5 and 75 mg/day.

In addition to the specific combinations disclosed herein, combinations made of an NMDA receptor antagonist such as an aminoadamantane compound and a second agent may be identified by testing the ability of a test combination to lessen the migraine or headache.

For a specified range a physician or other appropriate health professional will typically determine the best dosage for a given patient, according to his sex, age, weight, pathological state, and other parameters. In some cases, it may be necessary to use dosages outside of the ranges stated in pharmaceutical packaging insert to treat a subject. Those cases will be apparent to the prescribing physician or veterinarian.

In some embodiments, the combinations of the invention achieve therapeutic levels while minimizing debilitating side-effects that are usually associated with immediate release formulations. Furthermore, as a result of the delay in the time to obtain peak plasma level and the potentially extended period of time at the therapeutically effective plasma level, the dosage frequency may be reduced to, for example, once or twice daily dosage, thereby improving patient compliance and adherence.

Accordingly, the combination of the invention allows the NMDA receptor antagonist and the second agent to be administered in a combination that improves efficacy and avoids undesirable side effects of both drugs. For example, side effects including psychosis and cognitive deficits associated with the administration of NMDA receptor antagonists may be lessened in severity and frequency through the use of controlled-release methods that shift the Tmax to longer times, thereby reducing the dC/dT of the drug. Reducing the dC/dT of the drug not only increases Tmax, but also reduces the drug concentration at Tmax and reduces the Cmax/Cmean ratio providing a more constant amount of drug to the subject being treated over a given period of time and reducing adverse events associated with dosing. Similarly, side effects associated with the use of beta adrenergic antagonists, serotonin antagonists, steroids, serotonin receptor agonists, verapamil, or botulinum toxin may also be reduced in severity and frequency through controlled release methods.

In certain embodiments, the combinations provide additive effects. Additivity is achieved by combining the active agents without requiring controlled release technologies. In other embodiments, particularly when the pharmacokinetic profiles of the combined active pharmaceutical ingredients are dissimilar, controlled release formulations optimize the pharmacokinetics of the active pharmaceutical agents to reduce the variability of the Cratio over time. Reduction of Cratio variability over a defined time period enables a concerted effect for the agents over that time, maximizing the effectiveness of the combination. The Cratio variability (“Cratio.var”) is defined as the standard deviation of a series of Cratios taken over a given period of time divided by the mean of those Cratios multiplied by 100%. The Cratio for the controlled release formulation is more consistent than for the IR administration of the same drug over any significant time period, including shortly after administration and at steady state.

### Modes of Administration

The combination of the invention may be administered in either a local or systemic manner or in a depot or sustained release fashion. The two agents may be delivered in an oral, transdermal or intranasal formulation. In a preferred embodiment, the NMDA receptor antagonist, the second agent of the combination, or both agents may be formulated to provide controlled, extended release (as described herein). For example, a pharmaceutical composition that provides controlled release of the NMDA receptor antagonist, the second agent, or both may be prepared by combining the desired agent or agents with one or more additional ingredients that, when administered to a subject, causes the respective agent or agents to be released at a targeted rate for a specified period of time. The two agents are preferably administered in a manner that provides the desired effect from the first and second agents in the combination. Optionally, the first and second agents are admixed into a single formulation before they are introduced into a subject. The combination may be conveniently sub-divided in unit doses containing appropriate quantities of the first and second agents. The unit dosage form may be, for example, a capsule or tablet itself or it can be an appropriate number of such compositions in package form. The quantity of the active ingredients in the unit dosage forms may be varied or adjusted according to the particular need of the condition being treated.

Alternatively, the NMDA receptor antagonist and the second agent of the combination may not be mixed until after they are introduced into the subject. Thus, the term “combination” encompasses embodiments where the NMDA receptor antagonist and the second agent are provided in separate formulations and are administered sequentially. For example, the NMDA receptor antagonist and the second agent may be administered to the subject separately within 2 days, 1 day, 18 hours, 12 hours, one hour, a half hour, 15 minutes, or less of each other. Each agent may be provided in multiple, single capsules or tablets that are administered separately to the subject. Alternatively, the NMDA receptor antagonist and the second agent are separated from each other in a pharmaceutical composition such that they are not mixed until after the pharmaceutical composition has been introduced into the subject. The mixing may occur just prior to administration to the subject or well in advance of administering the combination to the subject.

If desired, the NMDA receptor antagonist and the second agent may be administered to the subject in association with other therapeutic modalities, e.g., drug, surgical, or other interventional treatment regimens. Accordingly, the combination described herein may be administered simultaneously or within 14 days, 7 days, 5 days, 3 days, one day, 12 hours, 6 hours, 3 hours, or one hour of additional therapeutic modalities. Where the combination includes a non-drug treatment, the non-drug treatment may be conducted at any suitable time so long as a beneficial effect from the co-action of the combination and the other therapeutic modalities is achieved. For example, in appropriate cases, the beneficial effect is still achieved when the non-drug treatment is temporally removed from the administration of the therapeutic agents, perhaps by days or even weeks.

### Formulations for Specific Routes of Administration

Combinations can be provided as pharmaceutical compositions that are optimized for particular types of delivery. For example, pharmaceutical compositions for oral delivery are formulated using pharmaceutically acceptable carriers that are well known in the art. The carriers enable the agents in the combination to be formulated, for example, as a tablet, pill, capsule, solution, suspension, powder, liquid, or gel for oral ingestion by the subject.

Alternatively, the compositions of the present invention may be administered transdermally via a number of strategies, including those described in U.S. Pat. Nos. 5,186,938, 6,183,770, 4,861,800 and WO 89/09051.

Pharmaceutical compositions containing the NMDA receptor antagonist and/or second agent of the combination may also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage may be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

In some embodiments, for example, the composition may be delivered intranasally to the cribriform plate rather than by inhalation to enable transfer of the active agents through the olfactory passages into the CNS and reducing the systemic administration. Devices used for this route of administration are included in U.S. Pat. No. 6,715,485. Compositions delivered via this route may enable increased CNS dosing or reduced total body burden reducing systemic toxicity risks associated with certain drugs.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The combination may optionally be formulated for delivery in a vessel that provides for continuous long-term delivery, e.g., for delivery up to 30 days, 60 days, 90 days, 180 days, or one year. For example the vessel can be provided in a biocompatible material such as titanium. Long-term delivery formulations are particularly useful in subjects with chronic conditions, for assuring improved patient compliance, and for enhancing the stability of the combinations. Formulations for continuous long-term delivery are provided in, e.g., U.S. Pat. Nos. 6,797,283; 6,764,697; 6,635,268, and 6,648,083.

If desired, the agents may be provided in a kit. The kit can additionally include instructions for using the kit. In some embodiments, the kit includes in one or more containers the NMDA receptor antagonist and, separately, in one or more containers, the second agent described herein (e.g., an opiate narcotic agent, a non-steroidal anti-inflammatory agent, or an anesthetic). In other embodiments, the kit provides a combination with the NMDA receptor antagonist and the second agent mixed in one or more containers.

The NMDA receptor antagonist, the second agent of the invention, or both agents may be provided in a controlled, extended release form. In one example, at least 50%, 90%, 95%, 96%, 97%, 98%, 99%, or even in excess of 99% of the NMDA receptor antagonist is provided in an extended release dosage form. A release profile, i.e., the extent of release of the NMDA receptor antagonist or the second agent over a desired time, may be conveniently determined for a given time by calculating the Cₘₐₓ/Cₘₑₐₙ for a desired time range to achieve a given acute or chronic steady state serum concentration profile. Thus, upon the administration to a subject (e.g., a mammal such as a human), the NMDA receptor antagonist has a Cmax/Cmean of approximately 2.5, 2, 1.5, or 1.0 approximately 1, 1.5, 2 hours to at least 6, 8, 9, 12, 18, 21, or 24 hours following such administration. If desired, the release of the NMDA receptor antagonist may be monophasic or multiphasic (e.g., biphasic). Moreover, the second agent may be formulated as an extended release composition, having a Cₘₐₓ/Cₘₑₐₙ of approximately 2.5, 2, 1.5, or 1.0, approximately 1, 1.5, 2 hours to at least 6, 8, 9, 12, 18, 21, 24 hours following administration to a subject. One of ordinary skill in the art can prepare combinations with a desired release profile using the NMDA receptor antagonists and the second agent and formulation methods known in the art or described below.

As shown in Tables 1 and 2, the pharmacokinetic half-lives of the drugs of both classes vary from about 1.5 hours to 70 hours. Thus, suitable formulations may be conveniently selected to achieve nearly constant concentration profiles over an extended period (preferably from 8 to 24 hours) thereby maintaining both agents in a constant ratio and concentration for optimal therapeutic benefits for both acute and chronic administration. Preferred Cratio,var values may be less than about 30%, 50%, 75%, 90% of those for IR administration of the same active pharmaceutical ingredients over the first 4, 6, 8, 12 hours after administration. Preferred Cratio,var values are less than about 100%, 70%, 50%, 30%, 20%, 10%.

Formulations that deliver this constant, measurable profile also allow one to achieve a monotonic ascent from an acute ratio to a desired chronic ratio for drugs with widely varying elimination half-lives. Compositions of this type and methods of treating patients with these compositions are embodiments of the invention. Numerous ways exist for achieving the desired release profiles, as exemplified below.

Suitable methods for preparing combinations in which the first agent, second agent, or both agents are provided in extended release-formulations include those described in U.S. Pat. No. 4,606,909 (hereby incorporated by reference). This reference describes a controlled release multiple unit formulation in which a multiplicity of individually coated or microencapsulated units are made available upon disintegration of the formulation (e.g., pill or tablet) in the stomach of the animal (see, for example, column 3, line 26 through column 5, line 10 and column 6, line 29 through column 9, line 16). Each of these individually coated or microencapsulated units contains cross-sectionally substantially homogenous cores containing particles of a sparingly soluble active substance, the cores being coated with a coating that is substantially resistant to gastric conditions but which is erodable under the conditions prevailing in the small intestine.

The combination may alternatively be formulated using the methods disclosed in U.S. Pat. No. 4,769,027, for example. Accordingly, extended release formulations involve prills of pharmaceutically acceptable material (e.g., sugar/starch, salts, and waxes) may be coated with a water permeable polymeric matrix containing an NMDA receptor antagonist and next overcoated with a water-permeable film containing dispersed within it a water soluble particulate pore forming material.

One or both agents of the combination may additionally be prepared as described in U.S. Pat. No. 4,897,268, involving a biocompatible, biodegradable microcapsule delivery system. Thus, the NMDA receptor antagonist may be formulated as a composition containing a blend of free-flowing spherical particles obtained by individually microencapsulating quantities of memantine, for example, in different copolymer excipients which biodegrade at different rates, therefore releasing memantine into the circulation at a predetermined rates. A quantity of these particles may be of such a copolymer excipient that the core active ingredient is released quickly after administration, and thereby delivers the active ingredient for an initial period. A second quantity of the particles is of such type excipient that delivery of the encapsulated ingredient begins as the first quantity's delivery begins to decline. A third quantity of ingredient may be encapsulated with a still different excipient which results in delivery beginning as the delivery of the second quantity beings to decline. The rate of delivery may be altered, for example, by varying the lactide/glycolide ratio in a poly(D,L-lactide-co-glycolide) encapsulation. Other polymers that may be used include polyacetal polymers, polyorthoesters, polyesteramides, polycaprolactone and copolymers thereof, polycarbonates, polyhydroxybuterate and copolymers thereof, polymaleamides, copolyaxalates and polysaccharides.

Optionally, the NMDA receptor antagonist, the second agent, or both agents are prepared using the OROS® technology, described for example, in U.S. Pat. Nos. 6,919,373, 6,923,800, 6,929,803, 6,939,556, and 6,930,128, all of which are hereby incorporated by reference. This technology employs osmosis to provide precise, controlled drug delivery for up to 24 hours and can be used with a range of compounds, including poorly soluble or highly soluble drugs. OROS® technology can be used to deliver high drug doses meeting high drug loading requirements. By targeting specific areas of the gastrointestinal tract, OROS® technology may provide more efficient drug absorption and enhanced bioavailability. The osmotic driving force of OROS® and protection of the drug until the time of release eliminate the variability of drug absorption and metabolism often caused by gastric pH and motility Alternatively, the combination may be prepared as described in U.S. Pat. No. 5,395,626 features a multilayered controlled release pharmaceutical dosage form. The dosage form contains a plurality of coated particles wherein each has multiple layers about a core containing an NMDA receptor antagonist and/or the second agent whereby the drug containing core and at least one other layer of drug active is overcoated with a controlled release barrier layer therefore providing at least two controlled releasing layers of a water soluble drug from the multilayered coated particle.

In some embodiments, the first agent and second agent of the combination described herein are provided within a single or separate pharmaceutical compositions. “Pharmaceutically or Pharmacologically Acceptable” includes molecular entities and compositions that do not produce an adverse, allergic or other untoward reaction when administered to an animal, or a human, as appropriate. “Pharmaceutically Acceptable Carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. “Pharmaceutically Acceptable Salts” include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The preparation of pharmaceutical or pharmacological compositions are known to those of skill in the art in light of the present disclosure. General techniques for formulation and administration are found in “Remington: The Science and Practice of Pharmacy, Twentieth Edition,”. Lippincott Williams & Wilkins, Philadelphia, Pa. Tablets, capsules, pills, powders, granules, dragees, gels, slurries, ointments, solutions suppositories, injections, inhalants and aerosols are examples of such formulations.

By way of example, extended release oral formulation can be prepared using additional methods known in the art. For example, a suitable extended release form of the either active pharmaceutical ingredient or both may be a matrix tablet composition. Suitable matrix forming materials include, for example, waxes (e.g., carnauba, bees wax, paraffin wax, ceresine, shellac wax, fatty acids, and fatty alcohols), oils, hardened oils or fats (e.g., hardened rapeseed oil, castor oil, beef tallow, palm dil, and soya bean oil), and polymers (e.g., hydroxypropyl cellulose, polyvinylpyrrolidone, hydroxypropyl methyl cellulose, and polyethylene glycol). Other suitable matrix tabletting materials are microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. Optionally, the finished tablet may be coated or uncoated.

The coating composition typically contains an insoluble matrix polymer (approximately 15-85% by weight of the coating composition) and a water soluble material (e.g., approximately 15-85% by weight of the coating composition). Optionally an enteric polymer (approximately 1 to 99% by weight of the coating composition) may be used or included. Suitable water soluble materials include polymers such as polyethylene glycol, hydroxypropyl cellulose, hydroxypropyl methyl cellulose, polyvinylpyrrolidone, polyvinyl alcohol, and monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups.

The coating composition may be plasticised according to the properties of the coating blend such as the glass transition temperature of the main agent or mixture of agents or the solvent used for applying the coating compositions. Suitable plasticisers may be added from 0 to 50% by weight of the coating composition and include, for example, diethyl phthalate, citrate esters, polyethylene glycol, glycerol, acetylated glycerides, acetylated citrate esters, dibutylsebacate, and castor oil. If desired, the coating composition may include a filler. The amount of the filler may be 1% to approximately 99% by weight based on the total weight of the coating composition and may be an insoluble material such as silicon dioxide, titanium dioxide, talc, kaolin, alumina, starch, powdered cellulose, MCC, or polacrilin potassium.

The coating composition may be applied as a solution or latex in organic solvents or aqueous solvents or mixtures thereof. If solutions are applied, the solvent may be present in amounts from approximate by 25-99% by weight based on the total weight of dissolved solids. Suitable solvents are water, lower alcohol, lower chlorinated hydrocarbons, ketones, or mixtures thereof. If latexes are applied, the solvent is present in amounts from approximately 25-97% by weight based on the quantity of polymeric material in the latex. The solvent may be predominantly water.

The pharmaceutical composition described herein may also include a carrier such as a solvent, dispersion media, coatings, antibacterial and antifungal agents, isotonic and absorption delaying agents. The use of such media and agents for pharmaceutically active substances is well known in the art. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition may also contain liquids, such as water, saline, glycerol, and ethanol, as well as substances such as wetting agents, emulsifying agents, or pH buffering agents. Liposomes, such as those described in U.S. Pat. No. 5,422,120, WO 95/13796, WO 91/14445, or EP 524,968 B1, may also be used as a carrier.

Additional methods for making controlled release formulations are described in, e.g., U.S. Pat. Nos. 5,422,123, 5,601,845, 5,912,013, and 6,194,000, all of which are hereby incorporated by reference.

Preparation for delivery in a transdermal patch can be performed using methods also known in the art, including those described generally in, e.g., U.S. Pat. Nos. 5,186,938 and 6,183,770, 4,861,800, and 4,284,444. A patch is a particularly useful embodiment in cases where the therapeutic agent has a short half-life. Patches can be made to control the release of skin-permeable active ingredients over a 12 hour, 24 hour, 3 day, and 7 day period. In one example, a 2-fold daily excess of an NMDA receptor antagonist is placed in a non-volatile fluid along with the opiate narcotic agent, non-steroidal anti-inflammatory agent, or anesthetic. Given the amount of the agents employed herein, a preferred release will be from 12 to 72 hours.

Transdermal preparations of this form will contain from 1% to 50% active ingredients. The compositions of the invention are provided in the form of a viscous, non-volatile liquid. Preferably, both members of the combination will have a skin penetration rate of at least 10⁻⁹ mole/cm²/hour. At least 5% of the active material will flux through the skin within a 24 hour period. The penetration through skin of specific formulations may be measures by standard methods in the art (for example, Franz et al., J. Invest. Derm. 64:194-195 (1975)).

In some embodiments, the composition may be delivered intranasally to the brain rather than by inhalation to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs.

Preparation of a pharmaceutical composition for delivery in a subdermally implantable device can be performed using methods known in the art, such as those described in, e.g., U.S. Pat. Nos. 3,992,518; 5,660,848; and 5,756,115.

Indications Suitable for Treatment with the Combination

Any subject experiencing or at risk of experiencing a migraine or headache may be treated as described herein. Additional conditions that may be treated using the combinations described herein include acute pain (e.g., post operative acute pain, low back pain, post-herpetic neuralgia, trigeminal neuralgia, spinal cord injury pain, carpal tunnel syndrome, cancer chemotherapy, phantom limb, ischemic pain, and pain due to burns), chronic pain (e.g., musculoskeletal pain, cancer pain, arthritis (including rheumatoid arthritis and osteoarthritis), pain resulting from sports injuries, back pain (such as low back pain), menstrual pain, gastrointestinal or urethral cramps, skin wounds or burns, and cancer pain.

Post operative acute pain and musculoskeletal chronic pain symptoms include any of the following: paraesthesias or dysaesthesias such as burning sensation, sharp pain, lightning pain, lancinating pain, paroxysmal pain, dull, achy pain, pins and needles sensation, referred pain, areas of the skin with diminished sensation, areas of heightened sensation, areas of abnormal sensation, reddened skin, skin hairs standing up, loss of hair, ulceration of skin, thinning of skin

Moreover, any CNS-related disorder, such as dementias (e.g., Alzheimer's disease, Parkinson's disease, Picks disease, fronto-temporal dementia, vascular dementia, normal pressure hydrocephalus, HD, and MCI), neuro-related conditions, dementia-related conditions, such as epilepsy, seizure disorders, acute pain, chronic pain, chronic neuropathic pain may be treated using the combinations and methods described herein. Epileptic conditions include complex partial, simple partial, partials with secondary generalization, generalized—including absence, grand mal (tonic clonic), tonic, atonic, myoclonic, neonatal, and infantile spasms. Additional specific epilepsy syndromes are juvenile myoclonic epilepsy, Lennox-Gastaut, mesial temporal lobe epilepsy, nocturnal frontal lobe epilepsy, progressive epilepsy with mental retardation, and progressive myoclonic epilepsy. The combinations of the invention are also useful for the treatment and prevention of pain caused by disorders including cerebrovascular disease, motor neuron diseases (e.g., ALS, Spinal motor atrophies, Tay-Sach's, Sandoff disease, familial spastic paraplegia), neurodegenerative diseases (e.g., familial Alzheimer's disease, prion-related diseases, cerebellar ataxia, Friedrich's ataxia, SCA, Wilson's disease, RP, ALS, Adrenoleukodystrophy, Menke's Sx, cerebral autosomal dominant arteriopathy with subcortical infarcts (CADASIL); spinal muscular atrophy, familial ALS, muscular dystrophies, Charcot Marie Tooth diseases, neurofibromatosis, von-Hippel Lindau, Fragile X, spastic paraplesia, psychiatric disorders (e.g., panic syndrome, general anxiety disorder, phobic syndromes of all types, mania, manic depressive illness, hypomania, unipolar depression, depression, stress disorders, PTSD, somatoform disorders, personality disorders, psychosis, and schizophrenia), and drug dependence (e.g., alcohol, psychostimulants (eg, crack, cocaine, speed, meth), opioids, and nicotine), Tuberous sclerosis, and Wardenburg syndrome), strokes (e.g, thrombotic, embolic, thromboembolic, hemmorhagic, venoconstrictive, and venous), movement disorders (e.g., PD, dystonias, benign essential tremor, tardive dystonia, tardive dyskinesia, and Tourette's syndrome), ataxic syndromes, disorders of the sympathetic nervous system (e.g., Shy Drager, Olivopontoicerebellar degeneration, striatonigral degeneration, PD, HD, Guillian Barre, causalgia, complex regional pain syndrome types I and II, diabetic neuropathy, and alcoholic neuropathy), Cranial nerve disorders (e.g., Trigeminal neuropathy, trigeminal neuralgia, Menier's syndrome, glossopharangela neuralgia, dysphagia, dysphonia, and cranial nerve palsies), myelopethies, traumatic brain and spinal cord injury, radiation brian injury, multiple sclerosis, Post-meningitis syndrome, prion diseases, myelitis, radiculitis, neuropathies (e.g., Guillian-Barre, diabetes associated with dysproteinemias, transthyretin-induced neuropathies, neuropathy associated with HIV, neuropathy associated with Lyme disease, neuropathy associated with herpes zoster, carpal tunnel syndrome, tarsal tunnel syndrome, amyloid-induced neuropathies, leprous neuropathy, Bell's palsy, compression neuropathies, sarcoidosis-induced neuropathy, polyneuritis cranialis, heavy metal induced neuropathy, transition metal-induced neuropathy, drug-induced neuropathy), axonic brain damage, encephalopathies, and chronic fatigue syndrome. Pain associated with any of these conditions may be treated using the methods and compositions described herein. All of the above disorders may be treated with the combinations described herein, whether pain is involved or not.

Immediate release formulations of memantine (e.g., Namenda) are typically administered at low doses (e.g., 5 mg/day) and progressively administered at increasing frequency and dose over time to reach a steady state serum concentration that is therapeutically effective. Namenda, an immediate release formulation of memantine, is first administered to subjects at a dose of 5 mg per day. After a period of time, subjects are administered with this dose twice daily. Subjects are next administered with a 5 mg and 10 mg dosing per day and finally administered with 10 mg Namenda twice daily. Using this dosing regimen, a therapeutically effective steady state serum concentration may be achieved within about thirty days following the onset of therapy. Using a sustained release formulation (22.5 mg) however, a therapeutically effective steady state concentration may be achieved substantially sooner, without using a dose escalating regimen. Such concentration is predicted to be achieved within 13 days of the onset of therapy. Furthermore, the slope during each absorption period for the sustained release formulation is less (i.e. not as steep) as the slope for Namenda. Accordingly, the dC/dt of the sustained release formulation is reduced relative to the immediate release formulation even though the dose administered is larger than for the immediate release formulation. Based on this model, a sustained release formulation of memantine may be administered to a subject in an amount that is approximately the full strength dose (or that effectively reaches a therapeutically effective dose) from the onset of therapy and throughout the duration of treatment. Accordingly, a dose escalation would not be required.

Treatment of a subject with the combination may be monitored using methods known in the art. If desired, treatment can be monitored by determining if the subject shows a decrease, in one or more of the descriptors associated with migraines: headaches, psychological symptomatology such as irritability, depression, fatigue, drowsiness, restlessness; neurological symptoms such as photophobia, phonophobia or gastrointestinal symptoms such as change in bowel habit, change of food intake or urinary symptoms such as urinary frequency, auras which are neurological deficits and can be a variety of deficits for the migraine population but in the individual is usually stereotyped. These deficits may be visual scotoma or visual designs, hemiplegia, migrating paraesthesia, dysarthria, dysphasia, or deja-vu. The headache is usually accompanied by light or sound sensitivity, photophobia or phonophobia, irritability and impaired concentration. A reduction in the following symptoms may also be observed: burning sensation, heat, cold, pressure, crushing, cramping, explosive, sharp pain, lightning pain, lancinating pain, stinging, knifelike, paroxysmal pain, dull, achy pain, pins and needles sensation, referred pain, areas of the skin with diminished sensation, areas of heightened sensation, areas of abnormal sensation, reddened skin, skin hairs standing up, loss of hair, ulceration of skin, thinning of skin. The efficacy of treatment using the combination is preferably evaluated by examining the subject's symptoms in a quantitative way, e.g., by noting a decrease in the frequency of attacks, or an increase in the time for sustained worsening of symptoms. In a successful treatment, the subject's status will have improved (i.e., frequency of relapses will have decreased, or the time to sustained progression will have increased).

The invention will be illustrated in the following non-limiting examples.

### Example 1

### In Vivo Method for Determining Optimal Steady-State Concentration Ratio (C_{ratio,ss})

A dose ranging study is performed using, for example, the vascular model (see, for example, Petty et al. Eur J Pharmacol 336: 127-36, 1997), the neurogenic model (see, for example, Petty et al. supra), the murine cutaneous allodynia model (see, for example, Ghelardini et al., J. Pain 5: 413-9, 2004), and the murine hyperalgesia model (see, for example, Galeotti et al., Pharmacol. Res. 46: 245-50, 2002). An isobolic experiment ensues in which the drugs are combined in fractions of their EDXXs to add up to ED100 (e.g., ED50:ED50 or ED25:ED75). The plot of the data is constructed. The experiment points that lie below the straight line between the ED50 points on the graph are indicative of synergy, points on the line are indicative of additive effects, and points above the line are indicative of inhibitory effects. The point of maximum deviation from the isobolic line is the optimal ratio. This is the optimal steady state ratio (Cratio,ss) and is adjusted based upon the agents half-life. Similar protocols may be applied in a wide variety of validated animal models.

### Example 2

### Combinations

Representative combination ranges and ratios are provided below for compositions of the invention. These ranges are based on the formulation strategies described herein.

### Adult Dosage and Ratios for Combination Therapy

| | Quantity, mg/day/(Second agent:NMDA Ratio Range) | | | | | | |
|---|---|---|---|---|---|---|---|
| NMDA drug | | | | | | | Dihydro- |
| mg/day | Propranolol | Verapamil | Methysergide | Sumatriptan | Frovatriptan | Eletriptan | ergotamine |
| Memantine/ | 40-240 | 45-480 | 0.5-10 | 7.5-100 | 0.25-7.5 | 5-40 | 0.25-4 |
| 2.5-80 | (0.5-96) | (0.56-192) | (0.006-4.0) | (0.09-40) | (0.003-3) | (0.06-16) | (0.003-1.6) |
| Amantadine/ | 40-240 | 45-480 | 0.5-10 | 7.5-100 | 0.25-7.5 | 5-40 | 0.25-4 |
| 50-400 | (0.1-4.8) | (0.11-9.6) | (0.001-0.2) | (0.019-2.0) | (0.0006-0.15) | (0.012-0.8) | (0.0006-0.08) |
| Rimantadine/ | 40-240 | 45-480 | 0.5-10 | 7.5-100 | 0.25-7.5 | 5-40 | 0.25-4 |
| 50-200 | (0.2-4.8) | (0.22-9.6) | (0.002-0.2) | (0.038-2.0) | (0.0013-0.15) | (0.025-0.8) | (0.0013-0.08) |

### Example 3

### Release Profile of Memantine and Dihydroergotamine

Release proportions are shown in the tables below for a combination of memantine and dihydroergotamine. The cumulative fraction is the amount of drug substance released from the formulation matrix to the serum or gut environment (e.g., U.S. Pat. No. 4,839,177) or as measured with a USP II Paddle system using water as the dissolution medium.

| | MEMANTINE | DIHYDROERGOTAMINE |
|---|---|---|
| | T½ = 60 hrs | T½ = 15 hrs |
| Time | cum. fraction A | cum. fraction B |
| 1 | 0.15 | 0.15 |
| 2 | 0.30 | 0.30 |
| 4 | 0.45 | 0.45 |
| 8 | 0.60 | 0.60 |
| 12 | 0.75 | 0.75 |
| 16 | 0.90 | 0.90 |
| 20 | 0.98 | 0.98 |
| 24 | 0.99 | 0.99 |

### Example 4

### Tablet Containing a Combination of Memantine and Frovatriptan

An extended release dosage form for administration of memantine and frovatriptan is prepared as three individual compartments. Three individual compressed tablets are prepared, each having a different release profile, are encapsulated into a gelatin capsule which is then closed and sealed. The components of the three tablets are as follows.

| Component | Function | Amount per tablet |
|---|---|---|
| TABLET 1 (immediate release): | | |
| Memantine | Active agent | 0 mg |
| Frovatriptan | Active agent | 1.0 mg |
| Dicalcium phosphate dihydrate | Diluent | 26.6 mg |
| Microcrystalline cellulose | Diluent | 26.6 mg |
| Sodium starch glycolate | Disintegrant | 1.2 mg |
| Magnesium Stearate | Lubricant | 0.6 mg |

| Component | Function | Amount per tablet |
|---|---|---|
| TABLET 2 (3-5 hour release): | | |
| Memantine | Active agent | 10 mg |
| Frovatriptan | Active agent | 1.0 mg |
| Dicalcium phosphate dihydrate | Diluent | 26.6 mg |
| Microcrystalline cellulose | Diluent | 26.6 mg |
| Sodium starch glycolate | Disintegrant | 1.2 mg |
| Magnesium Stearate | Lubricant | 0.6 mg |
| Eudragit RS30D | Delayed release | 4.76 mg |
| Talc | Coating component | 3.3 mg |
| Triethyl citrate | Coating component | 0.95 mg |

| Component | Function | Amount per tablet |
|---|---|---|
| TABLET 3 (Release delayed | | |
| 7-10 hours): | | |
| Memantine | Active agent | 12.5 mg |
| Frovatriptan | Active agent | 0.5 mg |
| Dicalcium phosphate dihydrate | Diluent | 26.6 mg |
| Microcrystalline cellulose | Diluent | 26.6 mg |
| Sodium starch glycolate | Disintegrant | 1.2 mg |
| Magnesium Stearate | Lubricant | 0.6 mg |
| Eudragit RS30D | Delayed release | 6.5 mg |
| Talc | Coating component | 4.4 mg |
| Triethyl citrate | Coating component | 1.27 mg |

The tablets are prepared by wet granulation of the individual drug particles and other core components as may be done using a fluid-bed granulator, or are prepared by direct compression of the admixture of components. Tablet 1 is an immediate release dosage form, releasing the active agents within 1-2 hours following administration. It contains no memantine to avoid the dC/dT effects of the current dosage forms. Tablets 2 and 3 are coated with the delayed release coating material as may be carried out using conventional coating techniques such as spray-coating or the like. The specific components listed in the above tables may be replaced with other functionally equivalent components, e.g., diluents, binders, lubricants, fillers, coatings, and the like.

Oral administration of the capsule to a patient will result in a release profile having three pulses, with initial release of frovatriptan from the first tablet being substantially immediate, release of the memantine and frovatriptan from the second tablet occurring 3-5 hours following administration, and release of the memantine and frovatriptan from the third tablet occurring 7-9 hours following administration.

### Example 5

### Beads Containing a Combination of Memantine and Propranolol Hydrochloride

The method of Example 4 is repeated substituting propranolol HCl for frovatriptan and using drug-containing beads in place of tablets. A first fraction of beads is prepared by coating an inert support material such as lactose with the drug which provides the first (immediate release) pulse. A second fraction of beads is prepared by coating immediate release beads with an amount of enteric coating material sufficient to provide a drug release-free period of 3-7 hours. A third fraction of beads is prepared by coating immediate release beads having half the ibuprofen dose of the first fraction of beads with a greater amount of enteric coating material, sufficient to provide a drug release-free period of 7-12 hours. The quantities of propranolol in each of the bead types is adjusted from the previous example to provide in a unit dose of 60 mg propranolol HCl evenly divided among the three types of beads. The three groups of beads may be encapsulated as in Example 4, or compressed, in the presence of a cushioning agent, into a single pulsatile release tablet. Alternatively, three groups of drug particles may be provided and coated as above, in lieu of the drug-coated lactose beads.

### Example 6

### Dissolution and Plasma Profiles

Experimental dissolution profiles were obtained from a USP II Paddle system using water as the medium (FIG. 1A). Simulations for propranolol and frovatriptan were generated using the Gastro Plus Software Package v.4.0.2 (FIGS. 2A, 3A). The corresponding in vivo release profiles were obtained using the Gastro-Plus software package v.4.0.2 (FIGS. 1B-C, 2B-D, 3B-D).

Memantine component of the Matrix Tablet Formulation 6601 shown in FIG. 1.

| | |
|---|---|
| Memantine HCL (22.5 mg) | 13.51% |
| Avicel PH102 | 60.04% |
| Eudragit RS-30D (30% w/w | 15.37% |
| aqueous dispersion) | |
| HPMC K100M | 10.08% |
| Magnesium Stearate | 1.00% |
| Total Component Weight | 166.5 mg |

Memantine component of the Coated Tablet Formulation 6701 shown in FIG. 1.

| | |
|---|---|
| Memantine HCL (22.5 mg) | 13.21% |
| Avicel PH102 | 58.72% |
| Eudragit RS-30D (30% w/w | 15.03% |
| aqueous dispersion) | |
| HPMC K100M | 9.86% |
| Magnesium Stearate | 0.98% |
| Opadry ® Clear, (Formulation | 2.20% |
| YS-1-7006, Colorcon) | |
| Total Component Weight | 170.3 mg |

Memantine component of the Coated Tablet Formulation 6801 shown in FIG. 1.

| | |
|---|---|
| Memantine HCL (22.5 mg) | 12.77% |
| Avicel PH102 | 56.55% |
| Eudragit RS-30D (30% w/w | 14.48% |
| aqueous dispersion) | |
| HPMC K100M | 9.50% |
| Magnesium Stearate | 0.94% |
| Opadry ® Clear, (Formulation | 3.00% |
| YS-1-7006, Colorcon) | |
| Surelease ® Clear, (Formulation | 2.80% |
| E-7-19010, Colorcon) | |
| Total Component Weight | 176.2 mg |

Propranolol component of the Matrix Tablet Formulation Short shown in FIG. 2.

| | |
|---|---|
| Propranolol (120 mg) | 13.56% |
| Avicel PH102 | 60.04% |
| Eudragit RS-30D (30% w/w | 15.37% |
| aqueous dispersion) | |
| HPMC K100M | 10.08% |
| Magnesium Stearate | 1.00% |
| Total Component Weight | 885 mg |

Propranolol component of the Coated Tablet Formulation SR shown in FIG. 2.

| | |
|---|---|
| Propranolol (120 mg) | 13.21% |
| Avicel PH102 | 58.72% |
| Eudragit RS-30D (30% w/w | 15.03% |
| aqueous dispersion) | |
| HPMC K100M | 9.86% |
| Magnesium Stearate | 0.98% |
| Opadry ® Clear, (Formulation | 2.20% |
| YS-1-7006, Colorcon) | |
| Total Component Weight | 908.4 mg |

Propranolol component of the Coated Tablet Formulation Long shown in FIG. 2.

| | |
|---|---|
| Propranolol (120 mg) | 12.77% |
| Avicel PH102 | 56.55% |
| Eudragit RS-30D (30% w/w | 14.48% |
| aqueous dispersion) | |
| HPMC K100M | 9.50% |
| Magnesium Stearate | 0.94% |
| Opadry ® Clear, (Formulation | 3.00% |
| YS-1-7006, Colorcon) | |
| Surelease ® Clear, (Formulation | 2.80% |
| E-7-19010, Colorcon) | |
| Total Component Weight | 940 mg |

Frovatriptan component of the Matrix Tablet Formulation SR shown in FIG. 3.

| | |
|---|---|
| Frovatriptan (3 mg) | 2.50% |
| Avicel PH102 | 71.05% |
| Eudragit RS-30D (30% w/w | 15.37% |
| aqueous dispersion) | |
| HPMC K100M | 10.08% |
| Magnesium Stearate | 1.00% |
| Total Component Weight | 120 mg |

Frovatriptan component of the Coated Tablet Formulation Short shown in FIG. 3.

| | |
|---|---|
| Frovatriptan (3 mg) | 2.50% |
| Lactose NF | 47.50% |
| Microcrystalline Cellulose NF | 39.80% |
| HPMC K100M | 8.20% |
| Magnesium Stearate | 1.00% |
| Opadry ® Clear, (Formulation YS- | 1.00% |
| 1-7006, Colorcon) | |
| Total Component Weight | 120 mg |

Frovatriptan component of the Coated Tablet Formulation Linear shown in FIG. 3.

| | |
|---|---|
| Frovatriptan (3 mg) | 2.30% |
| Avicel PH102 | 69.63% |
| Eudragit RS-30D (30% w/w | 15.03% |
| aqueous dispersion) | |
| HPMC K100M | 9.86% |
| Magnesium Stearate | 0.98% |
| Opadry ® Clear, (Formulation | 2.20% |
| YS-1-7006, Colorcon) | |
| Total Component Weight | 130.4 mg |

Frovatriptan component of the Coated Tablet Formulation Long shown in FIG. 3.

| | |
|---|---|
| Frovatriptan (3 mg) | 2.18% |
| Avicel PH102 | 67.10% |
| Eudragit RS-30D (30% w/w | 14.48% |
| aqueous dispersion) | |
| HPMC K100M | 9.50% |
| Magnesium Stearate | 0.94% |
| Opadry ® Clear, (Formulation | 3.00% |
| YS-1-7006, Colorcon) | |
| Surelease ® Clear, (Formulation | 2.80% |
| E-7-19010, Colorcon) | |
| Total Component Weight | 137.6 mg |

### Example 7

### A Patch Providing Extended Release of Memantine and Frovatriptan

As described above, extended release formulations of an NMDA antagonist are formulated for topical administration. Memantine transdermal patch formulations are prepared as described, for example, in U.S. Pat. Nos. 6,770,295 and 6,746,689.

For the preparation of a drug-in-adhesive acrylate, 5 g of memantine and 1 g of frovatriptan are dissolved in 10 g of ethanol and this mixture is added to 20 g of Durotak 387-2287 (National Starch & Chemical, U.S.A.). The drug gel is coated onto a backing membrane (Scotchpak 1012; 3M Corp., U.S.A.) using a coating equipment (e.g., RK Print Coat Instr. Ltd, Type KCC 202 control coater). The wet layer thickness is 400 μm. The laminate is dried for 20 minutes at room temperature and then for 30 minutes at 40° C. A polyester release liner is laminated onto the dried drug gel. The sheet is cut into patches and stored at 2-8° C. until use (packed in pouches). The concentration of memantine in the patches ranges between 5.6 and 8 mg/cm², while frovatriptan ranges between 1.1 and 1.6 mg/cm². The nearly continuous infusion of the components provides a much more consistent Cratio over time maximizing the additive or synergistic effects of the combinations of the present invention to achieve the optimal therapeutic effects.

Additional embodiments are within the claims.

What is claimed is:

1. A pharmaceutical composition comprising:
(a) an NMDA receptor antagonist;
(b) a second agent, wherein said second agent is selected from the group consisting of a beta adrenergic antagonist, serotonin antagonist, steroid, serotonin receptor agonist, calcium channel blocker, and Botulinum toxin; and
(c) a pharmaceutically acceptable carrier.

2. The pharmaceutical composition of claim 1, wherein at least one of said NMDA receptor antagonist or said second agent is provided in an extended release dosage form.

3. The pharmaceutical composition of claim 1, wherein said NMDA receptor antagonist has a dC/dT less than about 80% of the rate for the IR formulation.

4-6. (canceled)

7. The pharmaceutical composition of claim 1, wherein the NMDA receptor antagonist is selected from the group consisting of memantine, amantidine, rimantidine, ketamine, eliprodil, ifenprodil, dizocilpine, remacemide, iamotrigine, riluzole, aptiganel, phencyclidine, flupirtine, celfotel, felbamate, neramexane, spermine, spermidine, levemopamil, dextromethorphan, dextrorphan, and pharmaceutically acceptable salts thereof.

8. The pharmaceutical composition of claim 1, wherein said second agent is a beta adrenergic antagonist.

9. The pharmaceutical composition of claim 8, wherein said beta adrenergic antagonist is selected from the group consisting of propranolol, atenolol, nadolol, and pharmaceutically acceptable salts thereof.

10. The method of claim 1, wherein said second agent is a serotonin receptor agonist.

11. The method of claim 10, wherein said serotonin receptor agonist is selected from the group consisting of frovatriptan, sumatriptan, zolmitriptan, rizatriptan, naratriptan, eletriptan, ergotamine, dihydroergotamine, and pharmaceutically acceptable salts thereof.

12. The pharmaceutical composition of claim 1, wherein said second agent is selected from the group consisting of serotonin antagonists, steroids, Botulinum toxin, and pharmaceutically acceptable salts thereof.

13-14. (canceled)

15. A method of preventing or treating a CNS-related disorder comprising administering to a subject in need thereof a therapeutically effective amount of:
(a) an NMDA receptor antagonist; and
(b) a second agent, wherein said second agent is selected from the group consisting of a beta adrenergic antagonist, serotonin antagonist, steroid, serotonin receptor agonist, calcium channel blocker, and Botulinum toxin.

16. The method of claim 15, wherein said CNS-related disorder is a headache, vascular headache, migraine headache, cluster headache, or migraine.

17. The method of claim 15, wherein said CNS-related disorder is pain.

18. The method of claim 15, wherein said CNS-related condition is Alzheimer's disease or Parkinson's disease.

19. (canceled)

20. The method of claim 15, wherein said NMDA receptor antagonist is provided in an extended release dosage form.

21. The method of claim 20, wherein said NMDA receptor antagonist is administered at a substantially identical daily dose.

22. (canceled)

23. The method of claim 19, wherein said NMDA receptor antagonist has a dC/dT less than about 80% of the rate for the IR formulation.

24-26. (canceled)

27. The method of claim 15, wherein said NMDA receptor antagonist is selected from the group consisting of memantine, amantidine, rimantidine, ketamine, eliprodil, ifenprodil, dizocilpine, remacemide, iamotrigine, riluzole, aptiganel, phencyclidine, flupirtine, celfotel, felbamate, neramexane, spermine, spermidine, levemopamil, dextromethorphan, dextrorphan, and pharmaceutically acceptable salts thereof.

28. The method of claim 27, wherein said NMDA receptor antagonist is memantine.

29. The method of claim 28, wherein the amount of memantine ranges between 10 and 80 mg per dose.

30. (canceled)

31. The method of claim 15, wherein said second agent is a beta adrenergic antagonist.

32. The method of claim 31, wherein said beta adrenergic antagonist is selected from the group consisting of propranolol, atenolol, nadolol, and pharmaceutically acceptable salts thereof.

33. The method of claim 15, wherein said second agent is a serotonin receptor agonist.

34. The method of claim 33, wherein said serotonin receptor agonist is selected from the group consisting of frovatriptan, sumatriptan, zolmitriptan, rizatriptan, naratriptan, eletriptan, ergotamine, dihydroergotamine, and pharmaceutically acceptable salts thereof.

35. The method of claim 34, wherein said serotonin receptor agonist is frovatriptan.

36. The method of claim 35, wherein the amount of frovatriptan ranges between 0.25 to 7.5 mg per dose.

37-44. (canceled)

45. A kit comprising:
(a) an NMDA receptor antagonist;
(b) a second agent, wherein said second agent is selected from the group consisting of a beta adrenergic antagonist, serotonin antagonist, steroid, serotonin receptor agonist, calcium channel blocker, and Botulinum toxin; and
(c) instructions for treating or preventing a migraine, cluster headache, or vascular headache.

46. The kit of claim 45, wherein said NMDA receptor antagonist and said second agent are formulated as a single formulation.
